(19) United States
(12) Patent Application Publication
(10) Pub. No.: US 2014/0152842 A1
(43) Pub. Date: Jun. 5, 2014

(54) WAVEFRONT SENSOR DEVICE

(71) Applicant: Leslie Charles Laycock, Chelmsford-Essex (GB)

(72) Inventor: Leslie Charles Laycock, Chelmsford-Essex (GB)

(73) Assignee: BAE SYSTEMS PLC, London, EN (GB)

(21) Appl. No.: 14/238,587

(22) PCT Filed: Aug. 13, 2012
(86) PCT No.: PCT/GB2012/051967

§ 371 Date: Feb. 12, 2014

(30) Foreign Application Priority Data
Aug. 16, 2011 (GB) 1114068.8

(51) Int. Cl. H04N 5/33

(52) U.S. Cl. CPC H04N 5/33
USPC 348/164

(57) ABSTRACT

This invention relates to an infra red wavefront sensor device (10) including: • a plurality of tapered coherent fibre optic image conduits (12a, 12b), wherein each image conduit has an input surface (14a, 14b) and a body portion (16a, 16b) that tapers to an output surface (18a, 18b) and is configured to transmit an infra-red image incident on the input surface (14a, 14b) to the output surface (18a, 18b), in which the input surfaces (14a, 14b) are substantially coplanar thereby defining an image plane; • a plurality of detector arrays (26), wherein each detector array has a plurality of detector cells which are responsive to infra-red radiation, and in which each detector array (26) is positioned so as to detect an infra-red image appearing at the output surface (18a, 18b) of the paired image conduit (12a, 12b) by producing an output signal; and • an image processor (28) responsive to the output signals of the detector arrays (26) and configured to combine said output signals to produce a composite image.

This invention relates to an infra-red wavefront sensor device and an associated method of performing wavefront sensing.

Wavefront sensors are known devices for measuring aberrations in a wavefront. Applications include lens testing, ophthalmology and imaging. Abberations resulting from atmospheric turbulence are a particular problem for imaging. Shack-Hartmann wavefront sensors (SHWFS) are known instruments which are probably the most flexible and well established of current wavefront sensing techniques. A SHWFS uses a lenslet array (also called a microlens array) comprising lenslets of the same focal length to sample the wavefront over the aperture of the optical system. Each lenslet focuses light onto a position-sensitive sensor array. Aberrations in the wavefront cause the focused light spots on the sensor to be displaced from their central position. These variations in position are detected and the wavefront aberration can be determined by known means. A key requirement with SHWFS systems is a fast frame rate from the sensors (typically greater than or equal to 1000 frames per second (fps)) in order to correctly follow atmospheric aberrations. In practice, this typically necessitates the use of a camera with a “windowing” capability that maximises speed by limiting resolution.

There is interest in providing wavefront sensing capabilities at infra-red wavelengths. Advantages include covertness and the ability to use eyesafe lasers for illumination. However, some otherwise desirable infra-red detector materials such as InGaAs can only currently be exploited in cameras with relatively modest frame speeds when operating at useful resolutions. For example, the Sensors Unlimited, Inc. model SU320KTSW-1.7 RT infra-red camera which utilises InGaAs sensitive technology is limited to a speed of less than 150 fps when operating at a resolution of 256×256 pixels.

Thus, there is a need for an infra-red active wavefront sensor which can improve frame speed without sacrificing resolution, at least to an acceptable extent. More generally, it would be desirable to improve upon the combination of frame speed and resolution achievable with infra-red wavefront sensors, for example by improving resolution without sacrificing frame speed, or even improving both frame speed and resolution.

The present invention, in at least some of its embodiments, addresses these needs and desires. Although the invention has particular utility with InGaAs and HgCdTe detector technologies, the approach can be applied to other detector technologies.

According to a first aspect of the invention there is provided an infra-red wavefront sensor device including:
a plurality of tapered coherent fibre optic image conduits, wherein each image conduit has an input surface and a body portion that tapers to an output surface and is configured to transmit an infra-red image incident on the input surface to the output surface, in which the input surfaces are substantially co-planar thereby defining an image plane;
a plurality of detector arrays, wherein each detector array has a plurality of detector cells which are responsive to infra-red radiation, and in which each detector array is positioned so as to detect an infra-red image appearing at the output surface of the paired infra-red conduit by producing an output signal; and
an image processor responsive to the output signals of the detector arrays and configured to combine said output signals to produce a composite image.

In this way it is possible to improve the frame speed of the device at a given resolution in comparison to a device which utilises a single detector array. Conversely, by using a plurality of detector arrays it is possible to provide enhanced resolution at a given frame speed. It is highly advantageous to use tapered coherent fibre optic image conduits to form the image plane and transmit the infra-red image to the detector arrays. This is because the taper results in sufficient space for the necessary connections to be made to the detector arrays. If instead the image plane was formed directly by the detector arrays, then the detector arrays would have to be arranged with gaps therebetween to permit the necessary connections to be made. This would result in a loss of data in the composite image. This potential problem can be overcome by using indirect transmission of the image to the detector arrays via tapered coherent fibre optic image conduits.

The detector arrays may be positioned so that a subset of the detector cells in each detector array are used to detect an infra-red image appearing at the output surface of the paired image conduit. This permits the wavefront sensor device to operate at an enhanced frame speed in comparison to the frame speed achievable when the detector array is used at its maximum resolution. In other words, the resolution of each detector array is downgraded from the highest possible resolution, permitting a higher frame speed to be utilised. However, the device provides a composite image drawn from a plurality of detector arrays. Therefore, the resolution of the composite image is greater than the resolution utilised in respect of each of the individual detector arrays.

Typically, the detector arrays are integrated circuits having semiconductor detector cells.

In one of the embodiments, at least one of the image conduits has a body portion which is bent so that the output surface defines a plane which is not parallel to a plane defined by the input surface. Bent tapered coherent fibre optic image conduits of this type are particularly useful in connection with wavefront sensitive devices which utilise large numbers of image conduits and detector arrays.

The input surfaces may be arranged in an array wherein each input surface is substantially contiguous with each adjacent input surface. In this way, the edges of adjacent input surfaces in the array arrangement abut or very nearly abut so as to leave only a small gap. This enables a complete image of an area to be obtained without recourse to image processing techniques such as image extrapolation.

The input surfaces may be square or rectangular. This enables the input surfaces to be conveniently arranged in a substantially contiguous array. It also enables the format of the array to be well matched with that of the sensor.

Tapered coherent fibre optic image conduits are known devices which can reduce (or magnify) an image. The fibre optics are precisely aligned (usually as a fused bundle) so that an image is accurately transmitted to the output surface. Infra-red active tapered coherent fibre optic image conduits are produced commercially by, e.g. Schott AG (55122 Mainz, Germany; www.schott.com).

The wavefront sensor may be a Shack-Hartmann device. However, other kinds of wavefront sensor devices might be contemplated.

The device may further include a lenslet array positioned in front of the image plane.

Typically, the image conduits are arranged in an array of m rows and n columns of image conduits, where m and n are both greater than or equal to 2. In general, m and n are both greater than or equal to 3. Arrays in which m and n are both less than or equal to 3 can be constructed using bent tapered coherent fibre optic image conduits. Arrays in which m and n are both greater than 3 typically require the use of a number of straight image conduits of the type described above.

Generally, the device is configured to operate at infra-red wavelengths in the range 0.75 to 3.0 μm. The device may operate in the near infra-red region (0.75 to 1.4 μm) or the short wavelength infra-red region (1.4 to 3.0 μm). The terms “near infra-red” and “short wave length infra-red” are commonly abbreviated as NIR and SWIR respectively.

The device may be configured to operate at infra-red wavelengths of 1.2 μm or greater. A preferred operational wavelength range is 1.2 to 3.0 μm, with 1.2 to 2.0 μm being particularly preferred. The invention finds particular utility at these wavelengths. This is because currently available infra-red cameras are quite limited in terms of frame speeds when operating at a reasonable resolution, such as 256×256 pixels. As a result, at least some of the currently available cameras which operate in these wavelength ranges do not provide a high enough frame speed for practical use in a wavefront sensor device which maintains a useful resolution. The present invention provides a system in which the detector array technology utilised in such cameras can be incorporated into a practical wavefront sensing device.

The detector arrays may include InGaAs and HgCdTe detector cells.

Typically, each detector array is positioned in close contact with the output surface of its paired image conduit in order to maintain image resolution. Typically, each detector array is positioned within a few microns of the output surface of its paired image conduit; a representative range is 0-3 microns.

According to a second aspect of the invention there is provided a method of performing wavefront sensing including the step of detecting aberrations in a wavefront using an infra-red wavefront sensor device of the type including:
a plurality of tapered coherent fibre optic image conduits, wherein each image conduit has an input surface and a body portion that tapers to an output surface and is configured to transmit an infra-red image incident on the input surface to the output surface, in which the input surfaces are substantially co-planar thereby defining an image plane;
a plurality of detector arrays, wherein each detector array has a plurality of detector cells which are responsive to infra-red radiation, and in which each detector array is positioned behind the output surface of a paired image conduit so as to detect an infra-red image appearing at the output surface of the paired image conduit by producing an output signal; and
an image processor responsive to the output signals of the detector arrays and configured to combine said output signals to produce a composite image.

Whilst the invention has been described above, it extends to any inventive combination of the features set out above, or in the following description, drawings or claims.

Wavefront sensor devices in accordance with the invention will now be described, by way of example only, with reference to the accompanying drawings, in which:

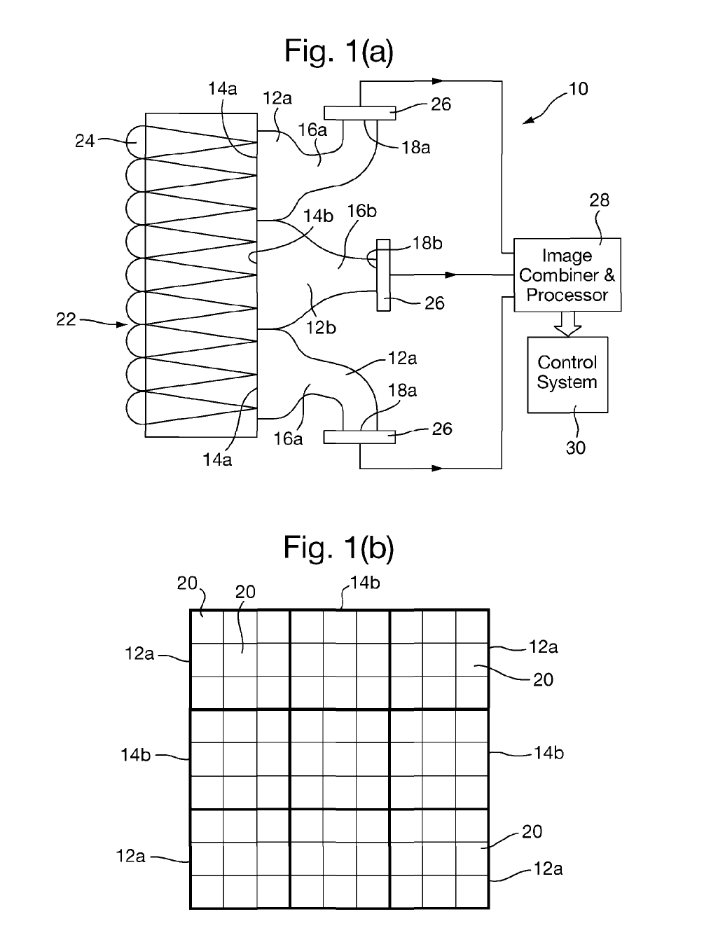

FIG. 1 shows (a) a semi-schematic side view of a wavefront sensor and (b) a front view of the image plane of the sensor; and

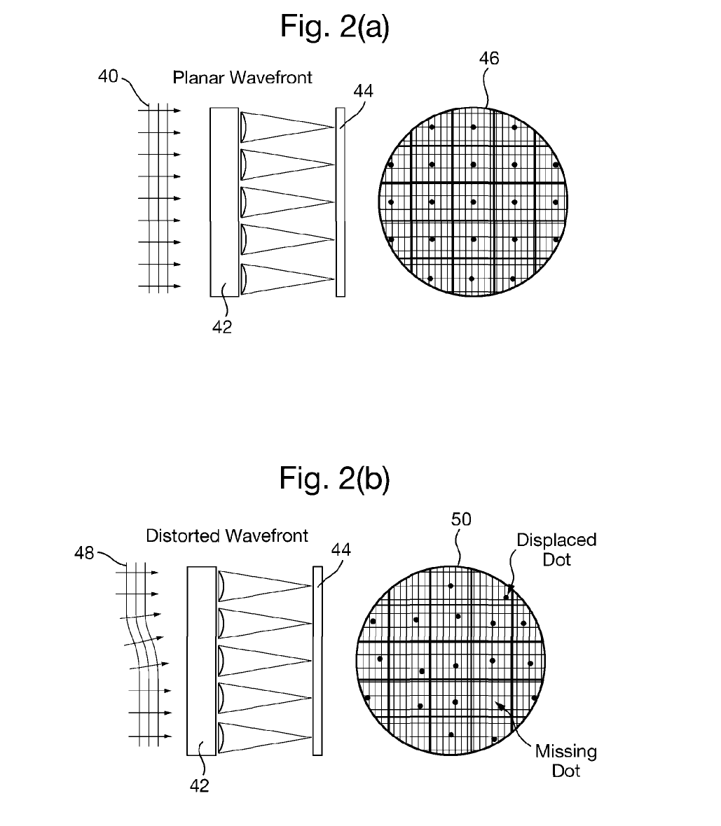

FIG. 2 shows the operation of a wavefront sensor with (a) an undistorted wavefront and (b) a distorted wavefront.

FIG. 1 shows an embodiment of a wavefront sensor device of the invention, depicted generally at 10, which comprises a plurality of tapered coherent fibre optic image conduits 12a, 12b. Each tapered coherent fibre optic image conduit comprises an input surface 14a, 14b, and a tapered body portion 16a, 16b which tapers towards an output surface 18a, 18b. The image conduits 12a are of the type having a bent body portion 16a, whereas the image conduits 12b are of the type having a straight body portion 16b. In the embodiment shown in FIG. 1, nine image conduits 12a, 12b are utilised in a 3×3 array. This can be seen to best effect in FIG. 1(b) which is an end view showing the input surfaces 14a, 14b of the image conduits 12a, 12b. Each image conduit is a bundle of tapered coherent fibre optics which is capable of transmitting an infra-red image from the input surface to the output surface. FIG. 1(b) shows the openings 20 of the fibre optics in each bundle. In FIG. 1(b) the narrow lines depict boundaries of the individual fibre optics, whereas the thick lines depict the boundaries of the image conduits. It will be appreciated by the skilled reader that for ease of presentation only nine fibre optics are shown in connection with each image conduit, but in practice a greater number of fibre optics may be present in each bundle. The input surfaces 14a, 14b in the 3×3 array define an image plane of the wavefront sensor device 10. It is highly convenient that the input surfaces 14a, 14b can abut one another to form an array wherein there are no gaps between adjacent input surfaces. This has the advantage that the array of input surfaces can transmit an essentially complete image.

The wavefront sensor device 10 further comprises a lenslet array 22. The lenslet array 22 comprises a plurality of lenslets or microlens 24 which are aligned with respect to the image plane so that the lenslets 24 focus infra-red radiation onto the input surfaces 14a, 14b. Each image conduit 12a, 12b is paired with a detector array 26, which is conveniently a chip or integrated circuit device having an array of infra-red detection cells formed thereon. Each detector array 26 is positioned against the output surface 18a, 18b of its paired image conduit 12a, 12b. In this way the detector array 26 detects the image which is transmitted along its paired image conduit. It is noted that the dimensions of the image conduits and detector arrays are chosen so that the surface area of the output surfaces of the image conduits is smaller than the active sensing area of the detector arrays. In other words, the image transmitted to the output surfaces of the image conduits is incident only on a subset of the detector cells in the detector array. This enables each detector array to be operated at a resolution which is less than the maximum resolution of the detector array. Alternatively the wavefront sensor can comprise of a number of smaller, less expensive detector arrays that operate at higher frame rates. The outputs of each detector array are fed to an image combiner and processor device 28. The image combiner and processor device 28 forms a mosaic of the images produced by each individual detector array so as to provide a composite image corresponding to the image incident on the image plane of the sensor device 10. The image combiner and processor device utilises components and image processing techniques such as distortion removal, which are well known to the skilled reader. The output of the image combiner and processor device 28 is fed to a control system 30 which may be of a type which is well known to the skilled reader, such as an adaptive optics system. Known techniques such as centroid tracking can be utilised to track any movement of the focused light spots on the detector cells and to use this information to correct for aberrations in the wavefront. In imaging applications, an illumination beam may be used to illuminate a target to provide signal for the waverfront sensor. Again, such techniques are well known in the art.

FIG. 2 is a diagrammatic representation of the operation of a wavefront sensor device of the type shown in FIG. 1. FIG. 2(a) shows the imaging of an undistorted, planar wavefront 40 using a wavefront sensor device of the type shown in FIG. 1. For presentational simplicity, FIG. 2 shows just the lenslet array 42 and the image plane 44 of the wavefront sensor device. As described above, the image plane 44 is made up of the aligned input surfaces of the tapered coherent fibre optic conduits utilised in the device. FIG. 2(a) shows also a portion 46 of the image produced by the wavefront sensor device. With an undistorted, planar wavefront 40 and an aligned lenslet array 42 and image plane 44, the infra-red radiation focused by the lenslets onto the image plane is observed as a series of focal spots positioned in the centre of each detector cell. FIG. 2(b) depicts an instance in which a distorted wavefront 48 is incident upon the lenslet array 42. The output of the wavefront sensor device for this instance is shown at 50, where it can be seen that aberrations in the distorted wavefront results in movement of the position of at least some of the focal spots on the detector cells. In some instances, the focal spot may be missing from its detector cell. The effect of this distortion can be countered using techniques which are well known in the art. However, in order to accurately follow atmospheric aberrations, it is necessary that the wavefront sensor device can be operated at a fast frame rate. Typically a frame rate of greater than or equal to 1000 frames per second is required. Current InGaAs chip sensors are quite limited in terms of frame speeds. For example, the Sensors Unlimited, Inc. model SU320KTSW-1.7RT camera is limited to a frame speed of less than 150 fps when operating at a resolution of 256×256 pixels. However, a frame speed of around 1700 fps can be achieved at a resolution of 64×64 pixels. This frame speed is suitable for use in infra-red wavefront sensor devices, but the associated resolution is not. The present invention enables InGaAs sensor technology to be utilised in wavefront sensor devices with acceptable frame speeds and resolution. This is achieved through using a plurality of sensor chips which are “tiled” together using tapered coherent fibre optic image conduits. In this way a composite image is obtained at an acceptable combination of frame speed and resolution. The use of tapered coherent fibre optic image conduits enables sufficient space to be created behind the image plane for the necessary interconnections to be made. If instead the detector arrays were tiled together on a substrate such as a PCB to provide the image plane of a device, then adjacent detector arrays would have to be spaced apart in order to provide sufficient room for interconnects. This would degrade the quality of the image produced by such a device. This potential problem is overcome using the tapered coherent fibre optic image conduits to provide the image plane of a wavefront sensor device.

Commercial available tapered coherent fibre optic image conduits and InGaAs based detector arrays can be used to produce a wavefront sensor device which can operate at wavelengths of 1.2 μm or greater. It is anticipated that future developments in tapered coherent fibre optic image conduits will improve performance at wavelengths in the SWIR and extend the range of operational infra-red wavelengths. Operation at wavelengths of 1.4 μm or greater is advantageous in view of eye safety, covertness and atmospheric considerations. However, the invention can be usefully applied at infra-red wavelengths below 1.2 μm. At infra-red wavelengths of around 1.0 μm or less, silicon sensor arrays can be used. Silicon detector arrays can be operated at much higher frame speeds than InGaAs sensor arrays. For example, for a silicon VNIR camera (Photonfocus 1024-160), a frame speed of greater than 2200 fps can be obtained at a resolution of 256×256 pixels. Nevertheless, the present invention can be used to improve image resolution at a given frame speed by utilising more than one silicon detector array. Also, if desired, the frame speed of wavefront sensor devices utilising silicon sensor arrays could be improved using the present invention to interrogate a subset of the available pixels on each detector array.

1. An infra-red wavefront sensor device including:
a plurality of tapered coherent fibre optic image conduits, wherein each image conduit has an input surface and a body portion that tapers to an output surface and is configured to transmit an infra-red image incident on the input surface to the output surface, in which the input surfaces are substantially co-planar thereby defining an image plane;
a plurality of detector arrays, wherein each detector array has a plurality of detector cells which are responsive to infra-red radiation, and in which each detector array is positioned so as to detect an infra-red image appearing at the output surface of the paired image conduit by producing an output signal; and
an image processor responsive to the output signals of the detector arrays and configured to combine said output signals to produce a composite image.

2. A device according to claim 1 in which the detector arrays are positioned so that a subset of the detector cells in each detector array are used to detect an infra-red image appearing at the output surface of the paired image conduit.

3. A device according to claim 1 in which the detector arrays are integrated circuits having semiconductor detector cells.

4. A device according to claim 1 in which at least one of the image conduits has a body portion which is bent so that the output surface defines a plane which is not parallel to a plane defined by the input surface.

5. A device according to claim 1 in which the input surfaces are arranged in an array wherein each input surface is substantially contiguous with each adjacent input surface.

6. A device according to claim 1 in which the input surfaces are square or rectangular.

7. A device according to claim 1 in which the wavefront sensor is a Shack-Hartmann device.

8. A device according to claim 1 further including a lenslet array positioned in front of the image plane.

9. A device according to claim 1 in which the image conduits are arranged in an array of m rows and n columns, where m and n are both greater than or equal to 2.

10. A device according to claim 9 in which m and n are both greater than or equal to 3.

11. A device according to claim 1 which is configured to operate at infra-red wavelengths in the range 0.75 to 3.0 μm.

12. A device according to claim 1 which is configured to operate at infra-red wavelengths in the range 1.2 μm or greater.

13. A device according claim 1 in which the detector arrays include InGaAs or HgCdTe detector cells.

14. A method of performing wavefront sensing including the step of detecting aberrations in a wavefront using an infra-red wavefront sensor device including:
a plurality of tapered coherent fibre optic image conduits, wherein each image conduit has an input surface and a body portion that tapers to an output surface and is configured to transmit an infra-red image incident on the input surface to the output surface, in which the input surfaces are substantially co-planar thereby defining an image plane;
a plurality of detector arrays, wherein each detector array has a plurality of detector cells which are responsive to infra-red radiation, and in which each detector array is positioned so as to detect an infra-red image appearing at the output surface of the paired image conduit by producing an output signal; and
an image processor responsive to the output signals of the detector arrays and configured to combine said output signals to produce a composite image.
